SEQUENCE LISTING (3 sequences)

SEQ ID NO: 1 - protein, 160 aa, Unknown
  WT (AM1_1557)
DIQDIFRATTHDVRESLGCDRVVVYKFFPDWSGEFLVEATAPNILPLSELEVPMVWQDTYLQENQGGKYRDNATTVVADIYQESYRDCHLEILEWYKIRAYMVVPVFIGETLWGLLAAYQLNVPRQWHKVELYLLKQAGAQLGVALQQAELLKQVRESRD

SEQ ID NO: 2 - protein, 160 aa, Unknown
  M10
DIQDIFRARTQDVRELLQADRVVVYRFNPDWSGEFVAEATAPNILPLSELEVPTVWEDTYLQENQGGKYRDNATTVVNDIYQESYRDCHLEILEWFQARAYMVVPIFQGETLWGLLAAYQNSAPRQWHKVELYLLKQLAGQLGVALQQAELLKQVRESRD

SEQ ID NO: 3 - protein, 160 aa, Unknown
  M13
DIQDIFRATTQDVRELLQCDRVVVYRFNPDWSGEFVAEATAPNILPLSELEVPTVWEDTYLQENQGGKYRDGATTVVNDIYQESYRDCHLEILEWFQARAYLVVPILQGETLWGLLAAYQNAAPRQWHKVELYLLKQLATQLGVALQQAELLKQVRESRD

(19) United States
(12) Patent Application Publication
(10) Pub. No.: US 2021/0202039 A1
(43) Pub. Date: Jul. 1, 2021

(54) METHOD FOR DESIGNING OPTIMIZED MUTANT PROTEIN SEQUENCE USING AMINO ACID COEVOLUTIONARY INFORMATION

(71) Applicant: KOREA INSTITUTE OF SCIENCE AND TECHNOLOGY, Seoul (KR)

(72) Inventors: Keunwan PARK, Gangneung-si (KR); Cheol-Ho PAN, Gangneung-si (KR); Moon-Hyeong SEO, Gangneung-si (KR); Dae-geun SONG, Gangneung-si (KR); Chulhee KWAK, Gangneung-si (KR)

(21) Appl. No.: 17/139,031

(22) Filed: Dec. 31, 2020

(30) Foreign Application Priority Data
Dec. 31, 2019 (KR) 10-2019-0179142

(51) Int. Cl. G16B 30/10

(52) U.S. Cl. CPC G16B 30/10

(57) ABSTRACT

The present invention relates to a method for designing an optimized mutant protein sequence using amino acid co-evolutionary information. Specifically, the present invention relates to a method for searching a protein having a novel mutant sequence with improved expression level, water solubility, stability, and functionality, while maintaining the original function of the protein, using amino acid co-evolutionary information and information on protein tertiary structure.

### CROSS-REFERENCE TO RELATED APPLICATION

This application claims priority to Korean Patent Application No. 10-2019-0179142, filed Dec. 31, 2019, and all the benefits accruing therefrom under 35 U.S.C. § 119, the content of which in its entirety is herein incorporated by reference.

### SEQUENCE LISTING

A Sequence Listing, incorporated herein by reference, is submitted in electronic form as an ASCII text file, created Dec. 30, 2020, of size 8 kB and named “8NV570202.txt”.

### TECHNICAL FIELD

The present invention relates to a method for designing an optimized mutant protein sequence using amino acid co-evolutionary information. Specifically, the present invention relates to a method for searching a protein having a novel mutant sequence with improved expression level, water solubility, stability, and functionality, while maintaining the original function of the protein, using amino acid co-evolutionary information and information on protein tertiary structure.

### BACKGROUND ART

Amino acid sequences and nucleic acid sequences are fundamental factors constituting proteins and genes, which are the basis of life phenomena. Therefore, for a biological application of proteins and genes, it is very important to improve a wide range of protein properties, such as expression, stability, etc., while maintaining the original functionality with respect to the amino acid or nucleic acid sequences.

Methods for searching important sites of amino acid or nucleic acid sequences include experimental methods and computational predictive methods. As the experimental methods, a method of artificially substituting an amino acid or nucleic acid at a target site, and then measuring energy changes, or examining changes in the actual functions thereof is mainly used. As the computational methods, a method for aligning evolutionarily-related amino acid or nucleic acid sequences through multiple sequence alignments and investigating whether each site is evolutionarily conserved is mainly used.

By analyzing the information about the alignment pattern of amino acids obtained when evolutionarily close protein sequences is aligned through multiple sequence alignments, not only conserved regions that are well conserved, but also co-evolved positions with similar patterns of mutation change can be found.

Co-evolution analysis method is an effective analysis method which is applied to find important sites having evolutionary variations. For example, as disclosed in Halabi, N. et al., 2009, Protein sectors: evolutionary units of three-dimensional structure. Cell, 138(4): 774-786, it is possible to search for important sites based on the evolutionary correlation between different positions.

That is, the co-evolutionary information provides information about position pairs that are closely located with each other in terms of the three-dimensional structure of the protein, or are functionally closely related.

However, there has been no report on methods of protein optimization with application of co-evolutionary information to protein engineering techniques.

Under these circumstances, the present inventors have made extensive efforts to develop a novel protein engineering method that can improve a wide range of proteins properties, such as protein expression level, water solubility, stability, functionality, etc., while maintaining the original functionality of the protein, using co-evolutionary information and information on protein tertiary structure. As a result, they have confirmed that a novel mutant sequence can be proposed, which can efficiently improve various properties of general functional proteins and the original functionality thereof, thereby completing the present invention.

### DISCLOSURE

### Technical Problem

An object of the present invention is to provide a method for designing an optimized mutant sequence, including calculating amino acid co-evolutionary information; and searching for a mutant protein sequence with the maximum co-evolution score.

Another object of the present invention is to provide an optimized mutant sequence searched by the method for designing an optimized mutant sequence above.

### Technical Solution

Hereinbelow, the present invention will be described in detail. Meanwhile, each of the explanations and exemplary embodiments disclosed herein can be applied to other explanations and exemplary embodiments. That is, all combinations of various factors disclosed herein belong to the scope of the present invention. Furthermore, the scope of the present invention should not be limited by the specific disclosure provided hereinbelow.

Additionally, those of ordinary skill in the art may be able to recognize or confirm, using only conventional experimentation, many equivalents to the particular aspects of the invention described herein. Furthermore, it is also intended that these equivalents be included in the present invention.

In order to achieve the objects of the present invention, an aspect of the present invention provides a method for designing an optimized mutant sequence, including calculating amino acid co-evolutionary information; and searching for a mutant sequence with the maximum co-evolution score.

As used herein, the “amino acid co-evolutionary information” refers to the information about position pairs which are closely located with each other in terms of the three-dimensional structure of the protein, or are functionally closely related.

By analyzing the information about the alignment pattern of amino acids obtained when evolutionarily close protein sequences is aligned through multiple sequence alignments, not only conserved regions that are well conserved, but also co-evolved positions with similar patterns of mutation change can be found.

The co-evolutionary information can be obtained from a technique for performing co-evolution analysis based on correlation by expressing amino acids or nucleic acids as numerical values such as substitution score matrix and measuring the correlation coefficient (Olmea, O. et al., 1999, Effective use of sequence correlation and conservation in fold recognition. Journal of Molecular Biology, 293(5): 1221-1239), a technique for performing co-evolution analysis based on SCA algorithm by changes in relative frequency of amino acid or nucleic acid pairs after randomly mixing multiple sequence alignments (Lockless, S. W. et al., 1999, Evolutionarily conserved pathways of energetic connectivity in protein families. Science, 286(5438): 295-299), or a technique based on mutual information, which is a co-evolution analysis method for measuring the frequency of amino acid or nucleic acid pairs as the degree of interdependence based on information theory (Atchley, W. R. et al., 2000, Correlations among amino acid sites in bHLH protein domains: an information theoretic analysis. Molecular biology and evolution, 17(1): 164-178), etc.

As used herein, the “method for designing an optimized mutant sequence” may be a protein optimization method based on the co-evolutionary information and information on protein tertiary structure.

The protein optimization method of the present invention may be performed in 6 steps including calculating amino acid co-evolutionary information; and searching for a mutant sequence with the maximum co-evolution score, but is not limited thereto.

The protein optimization method may include 1) searching for a functional position of a target protein; 2) fixing the sequence of the searched functional position, and then calculating co-evolutionary information of the amino acid sequence of the target protein; 3) identifying a functionally-relevant co-evolved position; 4) inducing a random mutation at the functionally-relevant co-evolved position; and 5) searching for a mutant sequence with the maximum co-evolution score; and 6) selecting a final candidate sequence and verifying protein properties of the selected sequence, but is not limited thereto.

As used herein, the “functional position” refers to an active position recognized as an important site for performing the original function of a target protein. That is, when the complex structure of protein-ligand, protein-protein, etc. is known, the binding interface of two materials may be defined as the functional position. Whereas, when the complex structure is not known, the functional position may include the position of “loss-of-function” mutation, the position of “gain-of-function” mutation, or “allosteric site”.

As used herein, the “functional position search” may consider a functional position predicted through a computational method when there is no known information about the functional position. In particular, available computational methods include ConSurf method (consurf.tau.ac.il/overview.php), which explores evolutionarily-conserved positions while being exposed to the outside, or PIRSitePredict method (research.bioinformatics.udel.edu/PIRSitePredict), which uses site-specific structure and experimental information, etc., but the computational methods are not limited thereto.

As used herein, the “important site” may be a position of an amino acid where it interacts mainly with a ligand or a protein, but is not limited thereto. That is, it may include all amino acid positions predicted to be functionally important.

In the present invention, the “important site” can be used interchangeably with the “important position”.

In one specific embodiment of the present invention, an amino acid position which is important for protein function, that is, the functional position of the target protein is searched, and then the amino acid position showing high co-evolutionary information when paired with the functional position can be identified.

In other words, the amino acid position having a high co-evolution score when paired with the functional position of the protein may be the functionally-relevant co-evolved position.

As used herein, the “co-evolutionary information” refers to numerically expressing the correlation of the mutation patterns of the amino acids for each position pair when the protein sequence information is aligned by multiple sequence alignment. The co-evolutionary information may be retrieved by calculating joint probability score of amino acid position pairs in the multiple sequence alignment information obtained from sequence information database, but is not limited thereto.

As used herein, the “sequence information database” refers to an online depository for storing amino acid or nucleic acid sequences, from which homologous sequences that are evolutionarily close to input sequences can be searched using an in-house program or an external program.

The sequence information database may include NCBI non-redundant database, and may further include GenBank, Uniprot, Protein Data Bank, etc. Additionally, the sequence search program may include PSI-BLAST, and the multiple sequence alignment program may include CLUSTAL-W, MUSCLE, etc., but the programs are not limited thereto. The PSI-BLAST may be used for homologous sequence search (Altschul, S. F. et al., 1997, Gapped BLAST and PSI-BLAST: a new generation of protein database search programs. Nucleic Acids Research, 25(17): 3389-3402), and the MUSCLE may be used for multiple sequence alignment (Edgar, R. C., 2004, MUSCLE: multiple sequence alignment with high accuracy and high throughput. Nucleic Acids Research, 32(5): 1792-1797).

As used herein, the “functionally-relevant co-evolved position” may include the information about the amino acid position having a high co-evolution score with the functional sequence position, but is not limited thereto.

As used herein, the “identification of the functionally-relevant co-evolved position” may be performed through all-versus-all co-evolution calculations for amino acid pairs. The PMRF program, Mip using mutual information, or SCA or Gremlin which analyzes statistical correlation, etc. may be used, but the method is not limited thereto.

As used herein, the “joint probability” refers to a numerical value expressing the degree of correlation of mutation patterns of amino acid or nucleic acid in each position pair based on the sequence information, and at the time, it refers to a calculated value for the probability of finding a specific amino acid pair. In particular, the calculation of the joint probability may be obtained using the preliminary information so that the multiple sequence alignment may be accurately carried out, but is not limited thereto.

As the preliminary information, a pseudocount, which corrects a small sample size, or a marginal probability may be used, but is not limited thereto.

If a multiple sequence alignment is used to calculate the joint probability between amino acid x at position i and amino acid y at position j of an unknown sequence, the probability of amino acid x at position i and the probability of amino acid y at position j may be multiplied and added to pseudocount (the same is applied for nucleic acids). Therefore, the corrected joint probability may be calculated by combining the joint probability value calculated from the multiple sequence without the pseudocount and the marginal probability value as a weighted sum. The pseudocount may be represented by the equation below:

P_{PP}(xᵢ,yⱼ)=(1−τ)P_{ML}(xᵢ,yⱼ)+τq(xᵢ)q(yⱼ)

wherein P_{PP}(xᵢ, yⱼ) is the corrected joint probability value to which the pseudocount based on a sequence profile is applied, P_{ML}(xᵢ, yⱼ) is the joint probability value to which the pseudocount is not applied, q(xᵢ) is the marginal probability of the amino acid or nucleic acid x at position i in the sequence profile, q(yⱼ) is the marginal probability of the amino acid or nucleic acid y at position j in the sequence profile, and i is the parameter value representing the weighted pseudocount, which may be predefined as a constant value or may be calculated to have a different value for each position based on the size of the multiple sequence alignments.

In the case where the marginal probability is constrained only to the information of the amino acids found in the multiple sequence alignment in order to calculate the joint probability between the amino acid x at position i and the amino acid y at position j of an unknown protein, it implies that a constraint condition in which the marginal probability calculated from the final joint probability coincides with the probability value obtained based on the multiple sequence alignment (the same is applied for nucleic acids). The marginal probability may be represented by the equation below:

$${\sum }_{y} {P}_{PA} ( {x}_{i} \cdot {y}_{j} ) = q ( {x}_{i} )$$

$${\sum }_{x} {P}_{P A} ( {x}_{i} \cdot {y}_{j} ) = q ( {y}_{j} )$$

wherein P_{PA}(xᵢ, yⱼ) is the joint probability score that the amino acids or nucleic acids x and y at positions i and j, respectively, appear at the same time, q(xᵢ) is the marginal probability of the amino acid or nucleic acid x at position i in the sequence profile, and q(yⱼ) is the marginal probability of the amino acid or nucleic acid y at position j in the sequence profile. Under these constraint conditions, the joint probability score can be optimized.

As the method for calculating co-evolution, for example, the pseudocount and the marginal probability may be used, but the method is not limited thereto.

In one specific embodiment of the present invention, the co-evolution score is a likelihood score for a sequence given by the joint probability and the marginal probability, and it may be calculated by Calculation Equation 1 below, but is not limited thereto.

$$P ( x ) = \frac{1}{z} {\prod }_{i} {q}_{i} ( {x}_{i} ) {\prod }_{i , j} {p}_{ij} ( {x}_{i} \cdot {x}_{j} ) \qquad [ Calculation Equation 1 ]$$

In the Equation above, P(x) represents the co-evolution score for a given sequence x, and Z represents the normalization factor for expressing probability values between 0 and 1.

The joint probability may provide information about the “functionally-relevant co-evolved position”, which shows a high co-evolutionary relationship with the position of the functional sequence. The co-evolved position refers to an amino acid position pair with evolutionarily similar mutation patterns. Random mutant sequences can be generated in a large scale at the co-evolved position. In particular, the optimized mutation position is constrained to the “functionally-relevant co-evolved position”, which has a high co-evolution score with the position of the functional sequence. Then, candidates for optimized mutant sequences can be searched among sequences which converge to the maximum co-evolution score.

The global optimization method, such as genetic algorithms, may be used to search for optimized mutant sequences of the present invention, but the method is not limited thereto. Sequence optimization by genetic algorithms creates a protein sequence population in which a random mutation has been generated in the “functionally-relevant co-evolved position”. The protein sequence population then generates various sequences through cross-over and mutation operation of the genetic algorithms, imparting priority to the sequences with high co-evolution scores. If the process is repeated until the co-evolution score no longer increases, only the sequences with high co-evolution scores survive, thereby reconstructing the protein sequence population.

As used herein, the “induction of mutation” refers to a computational replacement of amino acid A at a specific position with B. That is, it refers to performing a sequence substitution in a computational manner.

For the optimized mutant sequences which have finally survived, the structural stability of the sequences can be calculated through protein tertiary structure modeling and energy calculation. Mutant sequences with high co-evolution scores and high structural stability (low energy) are selected as final candidates. Finally, the protein optimization method may be terminated by verifying the degree of improvement of the functionality and the biochemical properties of the selected candidates for the optimized mutant sequences.

The co-evolved position is an amino acid position pair having high co-evolution scores, which can be calculated through the Z-score represented by Calculation Equation 2 below after obtaining a distribution of co-evolution scores for all amino acid position pairs, but is not limited thereto.

$$Z - score = \frac{x - μ}{σ} \qquad [ Calculation Equation 2 ]$$

In the Equation above, x is a specific co-evolution score, μ is the average value of the co-evolution scores, and σ is the standard deviation.

When the co-evolution score is calculated through the Z-score, a baseline is first established, and then an amino acid position pair having a co-evolution score higher than the baseline may be defined as a high co-evolution score. For example, the baseline may be 1.0, 1.2, or 1.5, but is not limited thereto.

In one specific embodiment of the present invention, the amino acid position pair having a value of 1.0 or more based on the Z-score was defined as having a high co-evolution score, which was used to define the functionally-relevant co-evolved position.

The high co-evolution score of the present invention can be defined by various methods in addition to the Z-score calculation. For example, the top 10% may be defined as a high co-evolution score, or the P-value may be calculated by a statistical permutation test to define a co-evolved position with a P-value of 0.01 or less as having a high co-evolution score, but the method is not limited thereto.

Additionally, a low energy value may be defined through Z-score represented by Calculation Equation 2 above by examining the energy value distribution of the entire mutant sequences, but is not limited thereto.

In one specific embodiment of the present invention, since a decrease in energy value indicates a higher stability, the low energy value was defined as having a value of −1.0 or less based on the Z-score.

The low energy value of the present invention may be defined by various methods in addition to the Z-score calculation. For example, the low energy value may be defined in consideration of ranking only, or the P-value may be calculated by a statistical permutation test to define a P-value of 0.01 or less as having a low energy value, but the method is not limited thereto.

As used herein, the “biochemical property” refers to the functionality, water solubility, thermal stability, or yield of a protein. As used herein, the “verification of the biochemical property” means verifying the functionality, water solubility, thermal stability, and protein yield. The “functional verification” may vary for different proteins. In the case of enzymes, the enzyme activity can be measured. In the case of a protein binding to a ligand, the binding affinity may be measured. In the case of a photoreactive protein, the property of binding to a chromophore may be measured. The “verification of water solubility” can measure the amount of protein through SDS-PAGE gel when the protein is obtained by being expressed in E. coli. That is, the solubility can be calculated by separately measuring the amount of protein in the soluble fraction and in the insoluble fraction. The “verification of thermal stability” can measure the temperature (melting temperature, Tm) at which the activity of the protein is lost by 50%. The Tm value can be measured using a circular dichroism device, but is not limited thereto. The “yield” refers to the amount of protein finally obtained by producing and purifying the protein.

As used herein, the “optimization” refers to an enhancement of biochemical properties, including water solubility, thermal stability, or yield, while maintaining the original functionality of a protein and thus having an improved functionality.

Another aspect of the present invention provides an optimized mutant sequence searched by the method for designing an optimized mutant sequence.

The terms “optimization” and “method for designing an optimized mutant sequence” of the present invention are as described above.

Although the optimized mutant sequence of the present invention is described as “a mutant sequence consisting of a specific amino acid sequence”, it is apparent that as long as the mutant sequence has an activity identical or corresponding to that of a protein consisting of an amino acid sequence of the corresponding sequence number, it does not exclude a mutation that may occur by a meaningless sequence addition upstream or downstream of the amino acid sequence, a mutation that may occur naturally, or a silent mutation thereof. Even when the sequence addition or mutation is present, it falls within the scope of the present invention.

For example, as long as the mutant sequence can perform the same or corresponding function as the protein molecule consisting of the amino acid, nucleic acid sequences showing a homology and/or identity of 85% or more, specifically 90% or more, more specifically 95% or more, even more specifically 98% or more, or even more specifically 99% or more to the sequence can also be included in the present invention. Additionally, it is obvious that an amino acid sequence with deletion, modification, substitution, or addition in part of the sequence also can be included in the scope of the present invention, as long as the amino acid sequence has such a homology.

As used herein, the term “homology” or “identity” refers to a degree of relevance between two given amino acid sequences, and may be expressed as a percentage. The terms “homology” and “identity” may often be used interchangeably with each other.

The sequence homology or identity of conserved polypeptide sequences may be determined by standard alignment algorithms and can be used with a default gap penalty established by the program being used. Substantially, homologous or identical sequences are generally expected to hybridize to all or at least about 50%, 60%, 70%, 80%, or 90% of the entire length of the sequences under moderate or high stringent conditions.

Whether any two polypeptide sequences have a homology, similarity, or identity may be determined by a known computer algorithm such as the “FASTA” program (Pearson et al., (1988) [Proc. Natl. Acad. Sci. USA 85]: 2444) using default parameters. Alternatively, it may be determined by the Needleman-Wunsch algorithm (Needleman and Wunsch, 1970, J. Mol. Biol. 48: 443-453), which is performed using the Needleman program of the EMBOSS package (EMBOSS: The European Molecular Biology Open Software Suite, Rice et al., 2000, Trends Genet. 16: 276-277) (preferably, version 5.0.0 or versions thereafter) (GCG program package (Devereux, J., et al., Nucleic Acids Research 12: 387 (1984)), BLASTP, BLASTN, FASTA (Atschul, [S.] [F.,] [ET AL., J MOLEC BIOL 215]: 403 (1990); Guide to Huge Computers, Martin J. Bishop, [ED.,] Academic Press, San Diego, 1994, and [CARILLO ETA/1 (1988) SIAM J Applied Math 48: 1073). For example, the homology, similarity, or identity may be determined using BLAST or ClustalW of the National Center for Biotechnology Information (NCBI).

The homology, similarity, or identity of polypeptides may be determined by comparing sequence information using, for example, the GAP computer program, such as Needleman et al. (1970), J Mol Biol. 48: 443 as disclosed in Smith and Waterman, Adv. Appl. Math (1981) 2:482. In summary, the GAP program defines the homology, similarity, or identity as the value obtained by dividing the number of similarly aligned symbols (i.e. amino acids) by the total number of the symbols in the shorter of the two sequences. Default parameters for the GAP program may include (1) a unary comparison matrix (containing a value of 1 for identities and 0 for non-identities) and the weighted comparison matrix of Gribskov et al. (1986), Nucl. Acids Res. 14:6745, as disclosed in Schwartz and Dayhoff, eds., Atlas of Protein Sequence and Structure, National Biomedical Research Foundation, pp. 353-358 (1979) or EDNAFULL substitution matrix (EMBOSS version of NCBI NUC4.4); (2) a penalty of 3.0 for each gap and an additional 0.10 penalty for each symbol in each gap (or a gap opening penalty of 10 and a gap extension penalty of 0.5); and (3) no penalty for end gaps.

Accordingly, as used herein, the term “homology” or “identity” refers to the relevance between sequences.

It was confirmed that the mutant protein searched by the method for designing an optimized mutant sequence of the present invention showed an increased expression level, water solubility, and thermostability as compared to a wild type.

In one specific embodiment of the present invention, it was confirmed that the protein consisting of the optimized mutant sequence finally selected as a candidate mutant sequence using the co-evolutionary information on AM1_1557g2 protein, which is one of the CBCR proteins, exhibited an increase in expression level, an increase in the amount of water-soluble proteins, and an increase in melting point, thereby increasing thermal stability (Examples 3 to 5).

From these results, it can be found that the protein optimization method using the amino acid co-evolutionary information of the present invention can provide a new mutant sequence that can efficiently improve the functionality of the protein.

### Advantageous Effects

The method for searching a protein having an optimized mutant sequence using amino acid co-evolutionary information of the present invention can design a new optimized protein sequence that can significantly improve protein yield, water solubility, thermal stability, and functionality, while maintaining the original function of the protein.

Therefore, the method for searching an optimized protein based on amino acid co-evolutionary information of the present invention can be applied to a wide range of application fields of functional proteins such as enzymes, biofuels, therapeutics, etc., and thus is expected to be useful for producing a protein having improved biochemical properties.

### BRIEF DESCRIPTION OF DRAWINGS

FIG. 1 is a diagram illustrating a process of forming an amino acid co-evolutionary network, which only consists of amino acid position pairs (functionally-relevant co-evolved position) having a high co-evolutionary relationship with functional positions based on protein structure information and sequence evolution information.

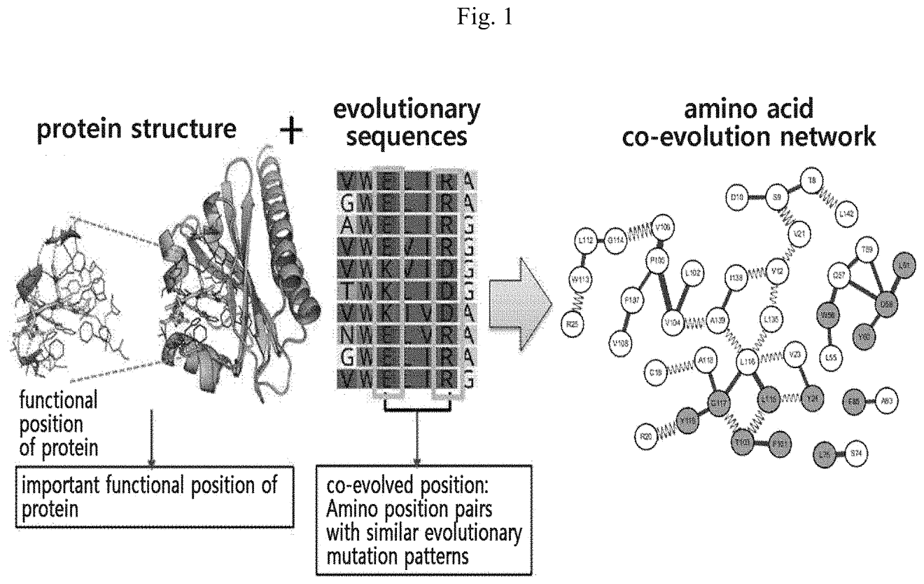

FIG. 2 is a diagram illustrating a global optimization algorithm for optimizing amino acid co-evolution scores.

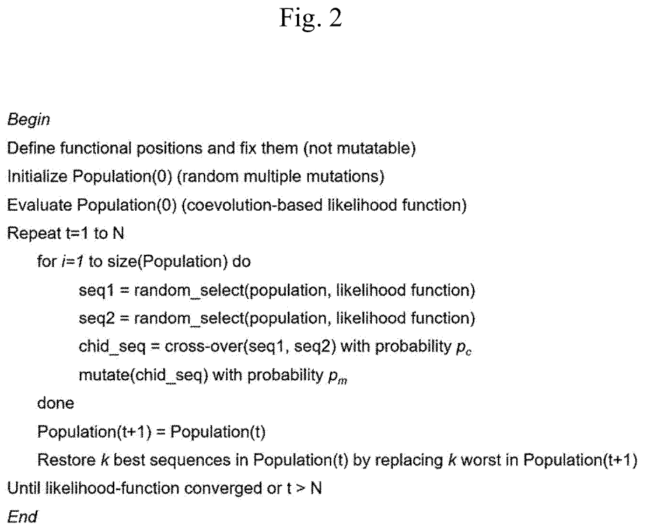

FIG. 3 is a diagram illustrating a process of searching a candidate sequence having optimized protein properties using amino acid co-evolutionary information.

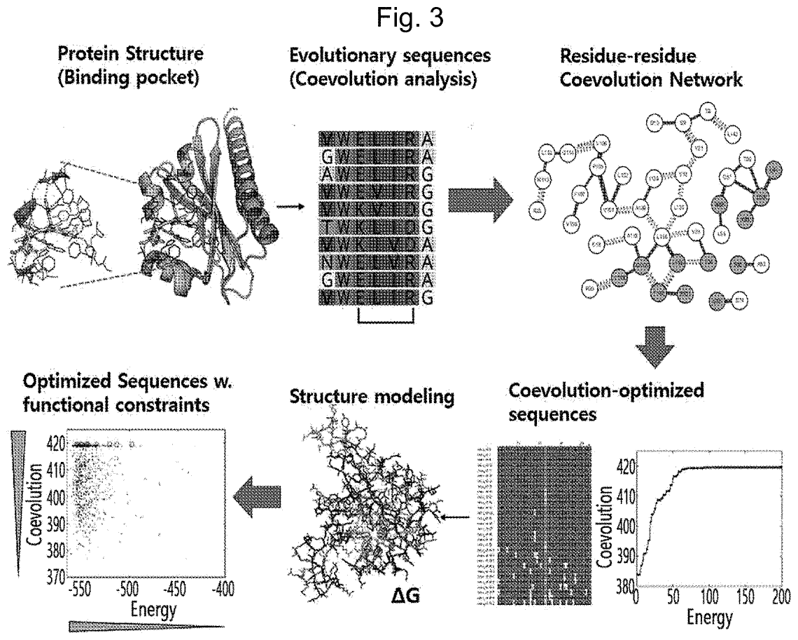

FIG. 4 is a structural diagram of the position of a functional sequence where the protein of interest AM1_1557g2 binds to a chromophore.

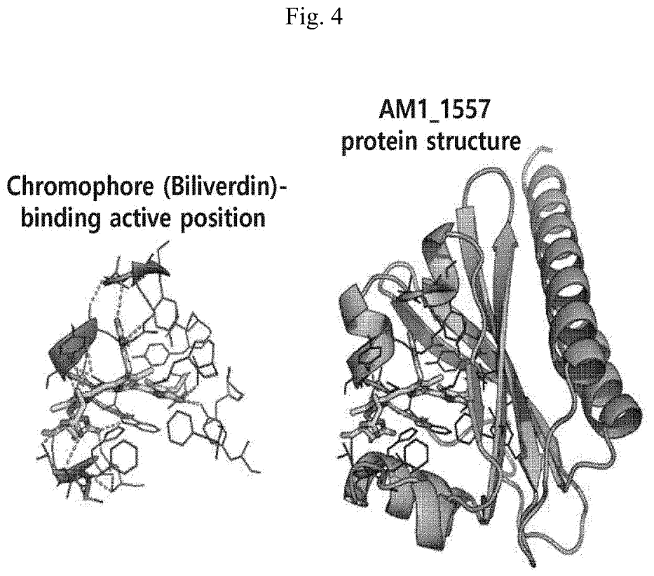

FIG. 5 is a diagram analyzing amino acid position pairs (functionally-relevant co-evolved position) having a high co-evolutionary relationship with a chromophore-binding position.

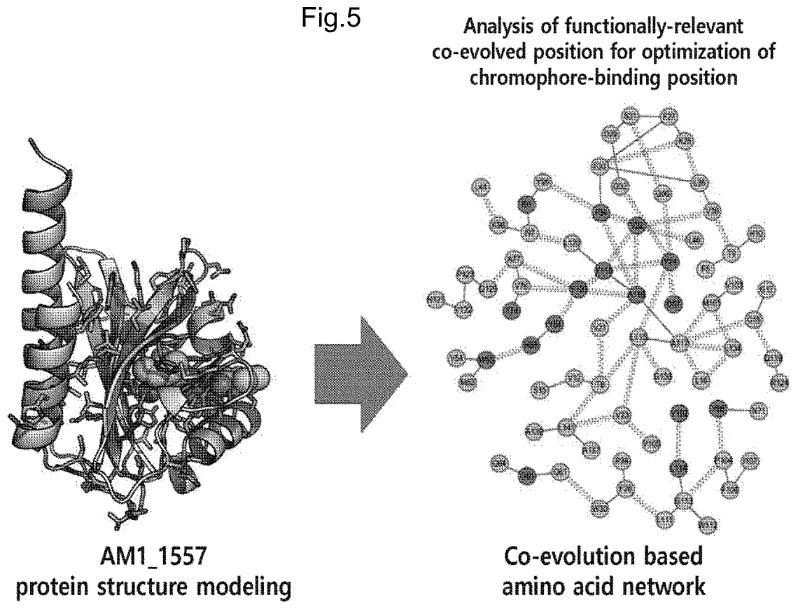

FIG. 6 is a diagram illustrating the result of searching candidate sequences having a high co-evolution score through global optimization of the functionally-relevant co-evolved position and performing energy calculation.

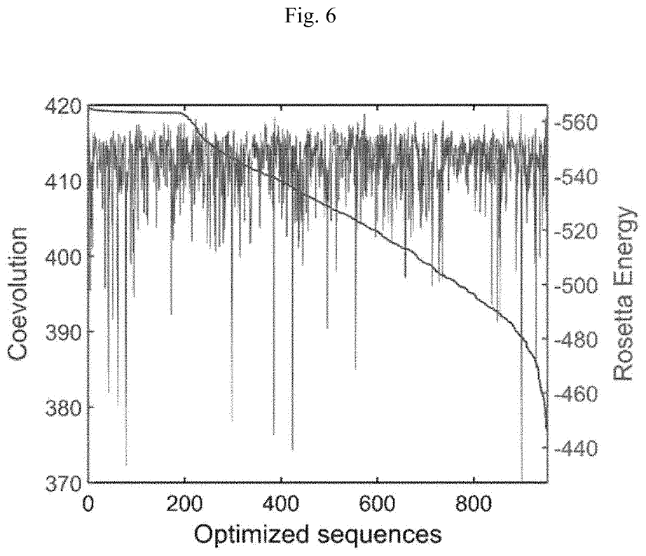

FIG. 7 is a diagram confirming the improved photoreactivity of the optimized protein.

FIG. 8 is a diagram confirming the increased expression level and water solubility of the optimized protein.

FIG. 9 is a diagram showing the improved thermal stability of the optimized protein.

### DETAILED DESCRIPTION OF PREFERRED EMBODIMENTS

The present invention will be described in more detail by way of Examples. However, these Examples are given for illustrative purposes only, and the scope of the invention is not intended to be limited to or by these Examples

### Example 1. Performing Protein Optimization Procedures

An optimized protein search using amino acid co-evolutionary information was performed as follows. The functional position of the protein was identified and the position of the functional sequence was fixed so that no mutation could be made. Subsequently, the amino acid co-evolutionary information was calculated and the “functionally-relevant co-evolved positions” showing high co-evolution with the functional positions was identified.

Calculation of the amino acid co-evolutionary information (co-evolution scores for amino acid pairs and for the entire sequence) was performed using the PMRF program (https://github.com/jeongchans/pmrf). The co-evolution calculation may be performed using SCA, Gremlin, or MISTIC program in addition to the PMRF program. The equation for calculating the co-evolution score for the sequence using the PMRF program used in the Examples is as follows.

$$P ( x ) = \frac{1}{z} {\prod }_{i} {q}_{i} ( {x}_{i} ) {\prod }_{i , j} {p}_{ij} ( {x}_{i} \cdot {x}_{j} ) \qquad [ Calculation Equation 1 ]$$

In the Equation above, P(x) represents the co-evolution score for a given sequence x, and Z represents the normalization factor for expressing probability values between 0 and 1.

Random mutant sequences were generated at the functionally-relevant co-evolved position in a large scale, and mutant sequences that converge to the maximum co-evolution score were searched through genetic algorithms.

Mutant candidate sequences with high co-evolution scores were selected, and the structural modeling and energy of the candidate sequences were calculated. After calculating the co-evolution scores and energy values of the candidate sequences, the final candidate sequences having a high co-evolution score and a low energy value were selected. The Rosetta program was used to calculate the energy value. Meanwhile, energy calculation programs such as Modeller, FoldX, AMBER, etc. can be used. The equation for energy calculation of the Rosetta program used in the Examples is as follows:

$$Δ {E}_{t o t a 1} = {\sum }_{i} {w}_{i} {E}_{i} ( {Θ}_{i} , {aa}_{i} )$$

| term | description | weight | units |
|---|---|---|---|
| fa_att | attractive energy between two [symbol] on different residues separated by a distance d | 1.0 | kcal/mol |
| fa_rep | rep [symbol] energy between two atoms on different residues separated by a distance d | 0.35 | kcal/mol |
| fa_int [symbol] _rep | rep [symbol] energy between two atoms on the same residue separated by a distance d | 0.005 | kcal/mol |
| [symbol] _ [symbol] l | Gaussian [symbol] implicit [symbol] energy between protein atoms in different residues | 1.0 | kcal/mol |
| lk_ball_ [symbol] td | orienta [symbol] -dependent s [symbol] lvation of polar atoms assuming ideal water geometry | 1.0 | kcal/mol |
| fa_intra_ [symbol] ol | Gaussian [symbol] implicit s [symbol] lvation energy between protein atoms in the same residue | 1.0 | kcal/mol |
| f [symbol] _ [symbol] iec | energy of interact [symbol] between two [symbol] charged atoms separated by a distance d | 1.0 | kcal/mol |
| hbond_lr_bb | energy of short-range hydrogen bonds | 1.0 | kcal/mol |
| hbond_sr_bb | energy of long-range hydrogen bonds | 1.0 | kcal/mol |
| hbond_bb_ [symbol] c | energy of backbone-side chain hydrogen bonds | 1.0 | kcal/mol |
| hbond_sc | energy of side chain-side chain hydrogen bonds | 1.0 | kcal/mol |
| delf_fal [symbol] | energy of [symbol] bridges | 1.25 | kcal/mol |
| rama_prep [symbol] o | probability of backbone ϕ, ψ angles given the amino acid type | (0.45 kcal/mol)/kT | kT |
| [symbol] _aa_pp | probability of amino acid identity given backbone ϕ, ψ angles | (0.4 kcal/mol)/kT | kT |
| fa_d [symbol] | probability that a chosen [symbol] is [symbol] -like [symbol] back [symbol] ϕ, ψ angles | (0.7 kcal/mol)/kT | kT |
| omega | backbone-dependant penalty for [symbol] [symbol] [symbol] that deviate from 0° and [symbol] ω [symbol] | (0.6 kcal/mol)/AU | AU [symbol] |
| | that deviate from 180° | | |
| pro_close | [symbol] for an open pro [symbol] ring and [symbol] ω bonding energy | (1.25 kcal/mol)/AU | AU |
| yhh_planarity | [symbol] penalty for [symbol] [symbol] χ, [symbol] angle | (0.625 kcal/mol)/AU | AU |
| ref | reference energies for amino acid types | (1.0 kcal/mol)/AU | AU |

[symbol]

AU = arbitrary units [symbol]
indicates data missing or illegible when filed

Then, the protein of the candidate sequences was secured, and the improvement of the biochemical properties of the protein was verified through experiments.

### Example 2. Optimization of Photoreactive Protein

Cyanobacteriochrome (CBCR), a phytochrome-photoreactive protein found in cyanobacteria, was used. The protein has various fluorescence spectra and mainly responds to light in the wavelength range of 530 nm to 660 nm.

That is, the functionality of the CBCR protein lies in photoreactivity, and CBCR essentially requires binding between a chromophore and the protein in order to carry out the photoreactive function as other phytochrome-based proteins.

The final candidate protein was selected by applying the optimization method of Example 1 using the co-evolutionary information on the AM1_1557g2 protein, which is one of the CBCR proteins.

Additionally, the results on the improvement of a wide range of protein properties were confirmed by verifying the expression level, water solubility, thermostability, and functionality of the candidate proteins.

First, the information of multiple sequence alignment for AM1_1557g2 was obtained using the GenBank non-redundant sequence database, which are databases for protein sequence and structure, and PSI-BLAST program.

The information on the functional sequence positions (chromophore-binding positions) was obtained through the protein tertiary structure modeling for AM1_1557g2.

Subsequently, the information on the positions which show a high co-evolutionary relationship with the binding position of the chromophore was obtained through the amino acid co-evolution analysis, and the functionally-relevant co-evolved positions were defined through the network analysis.

The candidate sequences with the highest co-evolution scores were searched among the mutant sequences at the functionally relevant co-evolved positions through genetic algorithms. That is, the sequences were sorted in the direction of increasing co-evolution score of the mutant sequences, and when the mutant sequences converged to the maximum evolution score as the optimization proceeded, an additional experimental verification was performed on the sequences having a high structural stability (low energy value) among the mutant sequences having high co-evolution scores.

The co-evolved position is an amino acid position pair having an evolutionarily high co-evolution score, which was calculated through Z-score represented by Calculation Equation 2 below after obtaining a distribution of co-evolution scores for all amino acid position pairs.

$$Z - score = \frac{x - μ}{σ} \qquad [ Calculation Equation 2 ]$$

In the Equation, x is a specific co-evolution score, μ is the average value of the co-evolution scores, and 6 is the standard deviation. The amino acid position pair having a value of 1.0 or more based on the Z-score was defined as having a high co-evolution score, which was used to define the functionally relevant co-evolved positions.

Additionally, the low energy value was defined through Z-score represented by Calculation Equation 2 above by examining the energy value distribution of the entire mutant sequences. Since a decrease in energy value indicates a higher stability, the low energy value was defined as having a value of −1.0 or less based on the Z-score.

### Example 3. Confirmation of Improved Photoreactivity of Optimized Protein

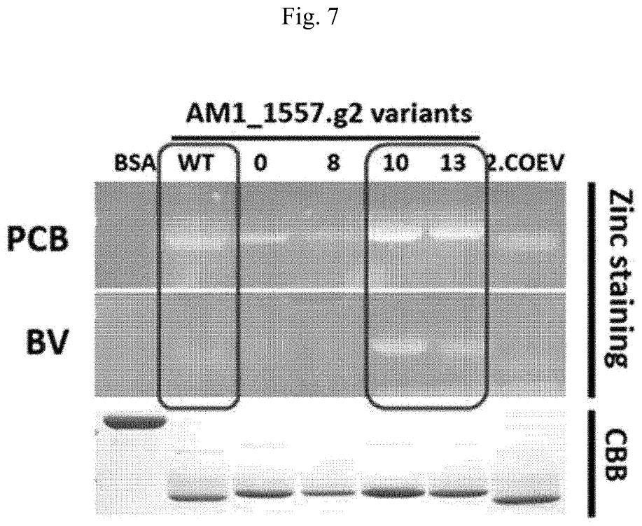

As a result of confirming the photoreactivity of the optimized protein, it was confirmed that the binding affinity of the chromophores was increased. That is, the degree of binding to the chromophores (PCB, BV) in the optimized mutant sequences was significantly increased compared to the control WT (wild type). In other words, the degree of binding to the chromophores can be determined through the sharpness of the band in zinc-blot assay, and it was confirmed that the sharpness of the band of the optimized mutant sequences (SEQ ID NOS: 10 and 13) was significantly increased (FIG. 7).

From these results, it was confirmed that the photoreactive protein consisting of the optimized mutant sequences designed by the method of the present invention showed an increase in binding affinity with the chromophores, indicating that the photoreactive functionality was enhanced.

### Example 4. Confirmation of Improved Expression Level and Increased Water Solubility of Optimized Protein

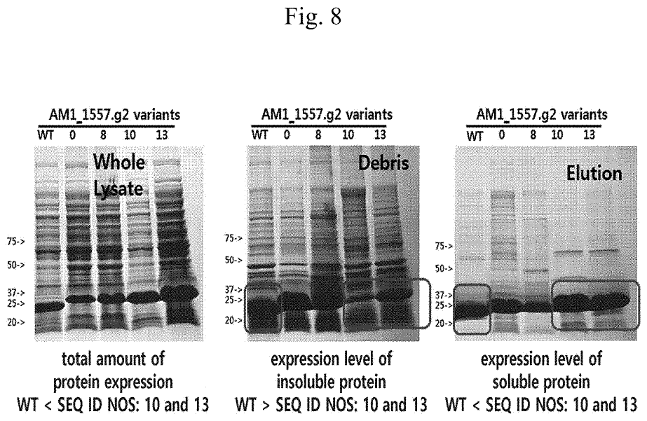

As a result of comparing the total amount of protein expression, it was confirmed that the protein expression level of the optimized mutant sequences was significantly increased compared to the wild type (WT, control) in Escherichia coli (FIG. 8).

Additionally, WT showed a higher expression level of insoluble proteins compared to the protein having the optimized mutant sequences (SEQ ID NOS: 10 and 13), while the protein having the optimized mutant sequences showed a higher expression level of soluble proteins compared to WT (FIG. 8). This indicates that the degree of water solubility of the protein having the optimized mutant sequences was significantly improved compared to WT.

From these results, the protein having the optimized mutant sequences showed a markedly increased expression level, suggesting that the degree of water solubility was significantly improved compared to the wild type.

### Example 5. Confirmation of Improved Thermal Stability of Optimized Protein

As a result of confirming the melting temperature through a circular dichroism experiment, it was confirmed that the melting point of the optimized mutant sequences was increased by 10° C. or higher.

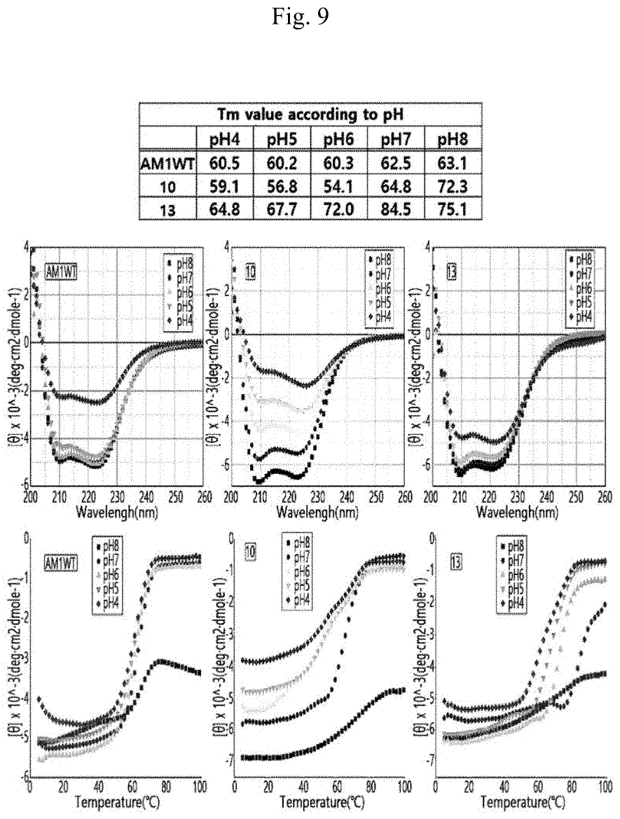

Specifically, the melting point of the wild-type protein was 63° C., while the melting point of the protein with the optimized mutant sequences (SEQ ID NOS: 10 and 13) was increased to around 80° C. (FIG. 9).

This indicates that the thermostability of the mutant sequences was significantly improved compared to WT (wild type).

The method for searching a protein having an optimized mutant sequence using amino acid co-evolutionary information of the present invention suggests that it is possible to design a novel optimized protein sequence that can significantly improve protein yield, water solubility and thermal stability, while maintaining the protein functionality.

From the above description, it will be understood by those skilled in the art to which the present invention pertains that the present invention may be embodied in other specific forms without departing from the technical spirit or essential characteristics of the present invention. In this regard, the embodiments described above are considered to be illustrative in all respects and not restrictive. Furthermore, the scope of the present invention is defined by the appended claims rather than the detailed description, and it should be understood that all modifications or variations derived from the meanings and scope of the present invention and equivalents thereof are included in the scope of the appended claims.

1. A method for designing an optimized mutant sequence, comprising
calculating amino acid co-evolutionary information; and
searching for a mutant sequence with the maximum co-evolution score.

2. The method of claim 1, wherein the method comprises:
searching for a functional position of a target protein;
identifying a functionally-relevant co-evolved position;
inducing a random mutation at the functionally-relevant co-evolved position; and
selecting a final candidate sequence and verifying protein properties of the selected sequence.

3. The method of claim 1, wherein the amino acid co-evolutionary information is retrieved by calculating a co-evolution score of amino acid position pairs in multiple sequence alignment information obtained from sequence information database.

4. The method of claim 3, wherein the sequence information database comprises NCBI non-redundant database.

5. The method of claim 3, wherein the multiple sequence alignment is obtained using PSI-BLAST or MUSCLE.

6. The method of claim 1, wherein the amino acid co-evolution score is calculated by the equation represented by Calculation Equation 1
$$P ( x ) = \frac{1}{z} {\prod }_{i} {q}_{i} ( {x}_{i} ) {\prod }_{i , j} {p}_{ij} ( {x}_{i} \cdot {x}_{j} ) \qquad [ Calculation Equation 1 ]$$
wherein P(x): Co-evolution score for a given sequence x, and Z: Normalization factor for expressing probability values between 0 and 1.

7. The method of claim 1, wherein the search for a mutant sequence with the maximum co-evolution score is performed using global optimization, which comprises genetic algorithms.

8. The method of claim 2, wherein the functionally-relevant co-evolved position comprises information on the amino acid position having a high co-evolution score with a functional sequence position, wherein the high co-evolution score has a value of 1.0 or higher based on the Z-score.

9. The method of claim 8, wherein the Z-score is calculated by the equation represented by Calculation Equation 2
$$Z - score = \frac{x - μ}{σ} , \qquad [ Calculation Equation 2 ]$$
wherein x: specific co-evolution score, μ: Average value of co-evolution scores, and σ: Standard deviation.

10. The method of claim 3, wherein the co-evolution score is calculated by using a pseudocount or marginal probability.

11. The method of claim 1, wherein the optimized mutant sequence is selected from sequences which converge to the maximum co-evolution score and have a low energy value, wherein the low energy has a value of −1.0 or less based on the Z-score.

12. The method of claim 11, wherein the selected optimized mutant sequence has an increased yield, expression level, water solubility, thermostability or functionality, compared to a wild type.

13. An optimized mutant sequence searched by the method for designing an optimized mutant sequence according to claim 1.
